(19) United States
(12) Patent Application Publication
(10) Pub. No.: US 2026/0114928 A1
(43) Pub. Date: Apr. 30, 2026

(54) ENHANCED VISIBILITY OF ABLATION DEVICES USING DOPPLER ULTRASOUND

(71) Applicant: Neuwave Medical, Inc., Madison, WI (US)

(72) Inventors: Jonathan DUFF, Madison, WI (US); Jason BRUNKOW, Mcfarland, WI (US)

(73) Assignee: Neuwave Medical, Inc., (omitted)

(21) Appl. No.: 18/928,992

(22) Filed: Oct. 28, 2024

(51) Int. Cl. A61B 34/20; A61B 18/00; A61B 18/18

(52) U.S. Cl. CPC A61B 34/20; A61B 18/1815; A61B 2018/00023; A61B 2018/00744; A61B 2018/1869; A61B 2034/2063

(57) ABSTRACT

A system is disclosed comprising an ablation probe and a controller. The ablation probe comprises a shaft including a stick region, an antenna extending from the shaft, and a cooling tube extending within the shaft. The controller is operable to convey first and second flow rates of coolant through the cooling tube. The first flow rate is sufficient to cause the ablation probe to vibrate, but insufficient to cause tissue to couple to the stick region. The second flow rate is sufficient to cause tissue to couple to the stick region. While conveying the first flow rate of coolant through the cooling tube, the controller is operable to emit, from an ultrasound transducer, a sound wave toward the ablation probe, receive, at the ultrasound transducer, a reflected sound wave from the ablation probe, and display, on a display, an image corresponding to the reflected sound wave.

### BACKGROUND

The present disclosure relates to ablation systems and, more particularly, to systems and methods for imaging ablation devices using doppler ultrasound.

Proper positioning of an ablation device within a patient is critical to ensuring that energy emitted therefrom affects intended targets, such as tumors, while avoiding healthy tissue and critical structures, such as arteries, as much as possible. The positioning of the ablation device may be determined using doppler ultrasound, which uses the relative speed of sound coming back to an ultrasound transducer to determine motion. Signals received at the transducer can, in turn, be used to determine a position of the ablation device. However, the echogenicity of ablation devices may be limited due to smooth surfaces thereof, which may reflect the sound waves away from the ultrasound transducer instead of scattering the sound waves back thereto.

Accordingly, systems and methods for improving the visualization of ablation devices using doppler ultrasound are desired.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an energy delivery system including an energy delivery device, a display, and an ultrasound transducer, in accordance with at least one aspect of the present disclosure.

FIG. 2 is a schematic diagram of an energy delivery device including a probe cannula and an antenna, in accordance with at least one aspect of the present disclosure.

FIG. 3 is an enlarged, cross-sectional view of the probe cannula and the antenna of FIG. 2, in accordance with at least one aspect of the present disclosure.

FIG. 4 is a schematic diagram of the probe cannula and the antenna of FIG. 3 percutaneously inserted into a patient and an ultrasound transducer positioned on the patient, in accordance with at least one aspect of the present disclosure.

FIG. 5 is an example view of the display of FIG. 1 displaying an image of the probe cannula and antenna of FIG. 4 based on sound waves received by the ultrasound transducer, in accordance with at least one aspect of the present disclosure.

FIG. 6 is an enlarged view of the probe cannula of FIG. 3 including a spring-loaded check valve attached to a distal end of a cooling tube, in accordance with at least one aspect of the present disclosure.

FIG. 7 is an enlarged view of the probe cannula of FIG. 3 including a reed attached to a distal end of a cooling tube, in accordance with at least one aspect of the present disclosure.

FIG. 8 is schematic flowchart of an example method of controlling the energy delivery system of FIG. 1, in accordance with at least one aspect of the present disclosure.

### DETAILED DESCRIPTION

The present disclosure is related to systems and methods for delivering energy to tissue for ablation operation and, more particularly, to systems and methods for graphically visualizing an expected ablation treatment area mid-procedure.

The present disclosure is related to comprehensive systems, devices, and methods for delivering energy (e.g., microwave energy, radiofrequency energy, laser, focused ultrasound, plasma, etc.) to tissue for a wide variety of applications including medical procedures (e.g., percutaneous or surgical). Example medical procedures that may benefit from the embodiments described herein include, but are not limited to, tissue ablation, resection, cautery, vascular thrombosis, intraluminal ablation of a hollow viscus, cardiac ablation for treatment of arrhythmias, electrosurgery, tissue harvest, cosmetic surgery, intraocular use, or any combination thereof.

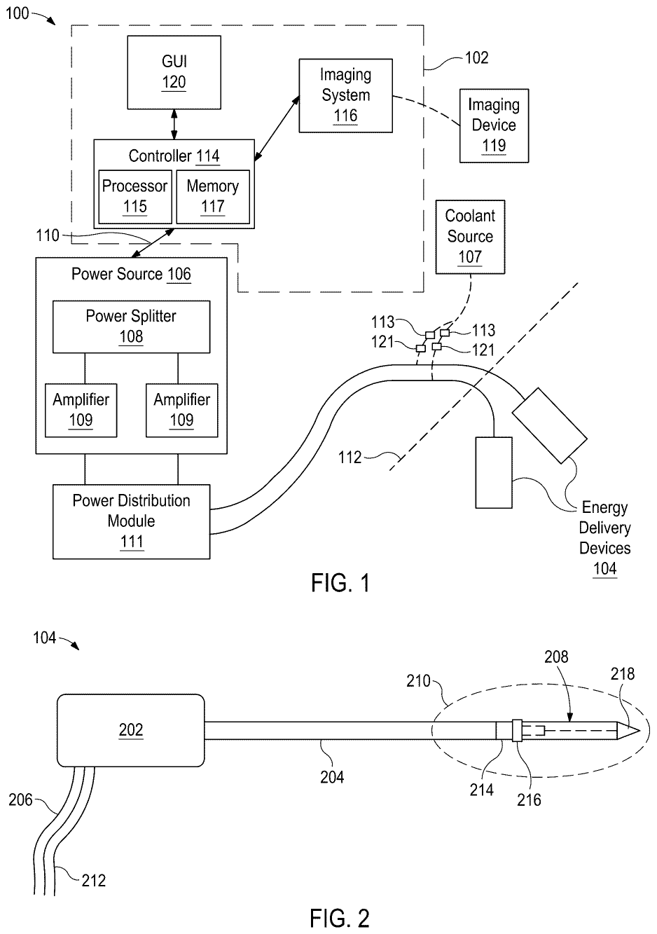

FIG. 1 is a block diagram of an energy delivery system 100, in accordance with at least one aspect of the present disclosure. As illustrated, the energy delivery system 100 (hereafter “the system 100”) may include a control system 102 and one or more energy delivery devices or “ablation probes” 104 (two shown) designed to deliver (emit) energy to a target tissue region of a patient. While two ablation probes 104 are shown, the system 100 may include only one ablation probe 104 or more than two ablation probes 104 (e.g. three, four, or five ablation probes).

The system 100 may further include a power source or generator 106 communicably coupled to the control system 102 and the ablation probes 104 to direct, control, and deliver (provide) electrical power thereto. The power source 106 may include a power splitter 108 that receives power from an external power source (e.g. a wall outlet) and directs power to one or more amplifiers 109 (two shown), which may amplify the voltage, current, or power from the power splitter 108 to an associated ablation probe 104. While two amplifiers 109 are shown, each associated with a corresponding ablation probe 104, the power source 106 may include less than two amplifiers (e.g. one amplifier) or more than two amplifiers (e.g. three, four, or five amplifiers, for example). Each amplifier 109 may be coupled to a corresponding ablation probe 104 via a power distribution module 111, which may provide strain relief to cabling extending from the amplifiers 109 to the ablation probes 104. The power distribution module 111 may be coupled to a structure in the operating room, such as a surgical bed, and may house connection hardware of the probes 104.

The power source 106 may supply energy required to operate various components of the system 100. The power source 106 may also supply energy to the ablation probes 104, such as microwave energy, radiofrequency energy, radiation, cryo energy, electroporation, high intensity focused ultrasound, or any combination thereof. In accordance with principles of the present disclosure, the power source 106 may supply microwave energy to the ablation probes 104 for purposes of tissue ablation. More specifically, power may be supplied to the ablation probes 104, but the microwave energy may be generated in a microwave generator and sent to the ablation probe 104. The power source 106 may include one or more energy generators configured to provide as much as 140-150 watts of microwave power at a frequency from 915 MHz to 5.8 GHz, although the present disclosure is not so limited. The power splitter 108 may comprise a power distribution system operable to distribute the energy from the power source 106 to the ablation probes 104. The power splitter 108 may be configured to provide varying energy levels to different regions of the ablation probes 104.

The control system 102 may monitor, control, and provide feedback concerning operation of the system 100. As illustrated, the control system 102 may include a controller 114, an imaging system 116, and a graphical user interface (GUI) or display 120, such as a touchscreen interface, which can be accessed by a user (e.g., a surgeon, a nurse, bedside assist, etc.) to operate the system 100. In some applications, the control system 102 may be mounted to or otherwise form part of a portable cart or “procedure cart,” and the GUI 120 may be arranged in a display region for operating and/or monitoring the components of the system 100.

The controller 114 may include a processor 115 and a memory or memory device 117 comprising any storage media readable by the processor 115. The memory 117 may store software or software instructions executable by the processor 115 to carry out functions and operations of the system 100. Examples of the memory 117 include, but are not limited to, random access memory (RAM), read-only memory (ROM), computer chips, optical discs (e.g., compact discs (CDs), digital video discs (DVDs), etc.), magnetic disks (e.g., hard disk drives (HDDs), floppy disks, ZIP® disks, etc.), magnetic tape, and solid state storage devices (e.g., memory cards, “flash” media, etc.). As used herein, the term “computer readable medium” refers to any device or system for storing and providing information (e.g., data and instructions) to the processor 115. Examples of computer readable media include, but are not limited to, optical discs, magnetic disks, magnetic tape, solid-state media, and servers for streaming media over networks.

Based on instructions provided by the software, the controller 114 may be configured to regulate the amount of energy (e.g., microwave energy) provided to a tissue region by the ablation probes 104 by monitoring characteristics of the tissue region, such as the size and shape of a target tissue, the temperature of the tissue region, etc. The controller 114 interacts with the ablation probes 104 to raise or lower (e.g., tune) the amount of energy delivered to the tissue region. The controller 114 may also be configured to prime coolants for distribution into the ablation probes 104 such that the coolant is delivered at a desired temperature, as discussed in more detail below.

In some applications, the type of tissue being treated is inputted into the software for purposes of allowing the controller 114 to regulate (e.g., tune) the delivery of microwave energy to the tissue region based upon pre-calibrated methods for that particular type of tissue or tissue region. In other embodiments, however, the type of probe selected for the particular procedure may be specifically tuned to a specific tissue type, and projected (expected) ablation sizes may be based on tissue type. In such embodiments, the controller 114 may not control power delivery based on tissue type. In yet other embodiments, the controller 114 generates a chart or diagram based upon a particular type of tissue or tissue region displaying characteristics useful to a user of the system.

The controller 114 may allow a user to choose power, duration of treatment, different treatment algorithms for different tissue types, simultaneous application of power to multiple probes 104, coherent and incoherent phasing, etc. The controller 114 may also be configured to create a database of information (e.g., required energy levels, duration of treatment for a tissue region based on particular patient characteristics, etc.) pertaining to ablation treatments for a particular tissue region based upon previous treatments with similar or dissimilar patient characteristics.

The imaging system 116 may be in communication with the controller 114 and comprise one or more imaging devices 119. Example imaging devices include, but are not limited to, ultrasound transducers, endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, or electromagnetic imaging. In some embodiments, the system 100 uses endoscopic cameras, imaging components, and/or navigation systems that permit or assist in placement, positioning, and/or monitoring of the ablation probes 104.

The imaging system 116 may be configured to monitor ablation procedures, such as a position of the ablation probes 104 within a patient, as described in more detail below, and/or the amount of ablation occurring within a particular tissue region(s) undergoing a thermal ablation procedure or. The monitoring includes, but is not limited to, MRI imaging, CT imaging, ultrasound imaging, nuclear medicine imaging, and fluoroscopy imaging. The software may be designed to automatically obtain images of a tissue region (e.g., MRI imaging, CT imaging, ultrasound imaging, nuclear medicine imaging, fluoroscopy imaging), automatically detect any changes in the tissue region (e.g., blood perfusion, temperature, amount of necrotic tissue, etc.), and based on the detection to automatically adjust the amount of energy delivered to the tissue region through the ablation probes 104.

The components of the system 100 may be connected via one or more cables or transmission lines 110. Moreover, the ablation probes 104 are designed to operate within a sterile field facilitated by the use of a sterile field barrier 112 that separates the ablation probes 104 from the remaining components of the system 100. The sterile field barrier 112 creates the sterile field, which includes any region permitting access only to sterilized items (e.g., sterilized devices, sterilized accessory agents, sterilized body parts, etc.). The sterile field barrier 112 hinders entry of non-sterile items into the sterile field, and the ablation probes 104 are configured for operation within the sterile field.

The system 100 may further include a coolant source 107, which stores therein a cooling fluid or “coolant”. Example coolants include, but are not limited to, water, glycol, air, inert gases (e.g., helium), carbon dioxide, nitrogen, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, or any combination thereof.

The system 100 may further include one or more valves 113 fluidically coupled to the coolant source 107 and a respective ablation probe 104. The valves 113 may control the flow rate and/or pressure of coolant from the coolant source 107 to the respective ablation devices 104. The valves 113 may be any suitable valve (e.g., gate, globe, ball, etc.) that are transitionable (actuatable) between an open state (e.g., 100% open), a closed state (e.g., 0% open), and a plurality of partially open states between the open and closed states (e.g., 10%, 25%, 50%, 75%, or 90%). In applications where the valves 113 are electromechanically actuatable, the valves 113 may each include a motor in operable communication with the controller 114, via a wired or wireless connection, and which function to transition the valves 113 between their respective open, closed, and partially open states. Alternatively, the valves 113 may be solenoid valves that are transitionable between their respective open, closed, and partially opened states by the controller 114. The valves 113 may be transitionable between their respective open, closed, and partially open states based on a user input provided to the GUI 120 or automatically, such as based on inputs provided to the controller 114 from various sensors of the system 100.

The system 100 may further include one or more sensors 121 for sensing one or more parameters associated with the coolant provided from the coolant source 107. The sensors 121 may include pressure sensors operable to sense a pressure of the coolant provided from the coolant source 107, flow sensors operable to sense a flow rate of the coolant provided from the coolant source 107, or temperature sensors for sensing a temperature of the coolant provided from the coolant source 107, or any combination thereof. Each valve 113 may have associated therewith one or more of the sensors 121 to sense one or more parameters of the coolant provided to the respective ablation probe 104. The controller 114 may be in operable communication with the one or more sensors 121, via a wired or wireless connection, and may receive the sensed parameters therefrom. The controller 114 may control one or more aspects of the system 100 based on the sensed parameters, such as the state of the valves 113.

FIG. 2 is a schematic diagram of an ablation probe 104 that may incorporate the principles of the present disclosure. As indicated above, the ablation probe 104 may be configured to deliver (emit) energy (e.g., microwave energy, radiofrequency energy, radiation energy) to a target tissue region. As illustrated, the ablation probe 104 includes a handle or housing 202 and an elongate shaft or probe cannula 204 extending distally from the handle 202.

A cable or cable assembly 206 may be operatively coupled to the handle 202 and configured to convey electrical power thereto. The cable assembly 206 may extend from the power distribution module 111 (FIG. 1), for example, and may provide the power sufficient to operate the ablation probe 104. An antenna 208 is provided at the distal end of the probe cannula 204 and receives electrical power from the cable assembly 206 to emit energy (e.g., microwave energy) to a target tissue region and thereby generate an ablation zone 210 (shown in dashed lines).

A cooling tube 212 may be operatively coupled to the coolant source 107 (FIG. 1) and the handle 202 and may be configured to convey the coolant from the coolant source 107 to the ablation probe 104.

The ablation probe 104 may include a sharp stylet tip or “stylet” 218 positioned at the distal end of the antenna 208 and otherwise forming the distal end of the ablation probe 104. The stylet 218 may facilitate percutaneous insertion of the ablation probe 104. The stylet 218 may be made of a variety of rigid or hardened materials including, but not limited to, a hardened resin, a metal (e.g., titanium or an equivalent of titanium, stainless steel, etc.), a ceramic, or any combination thereof. In at least one application, the stylet 218 may be brazed to zirconia or an equivalent of zirconia. In such applications, the stylet 218 may comprise an extension of a metal portion of the antenna 208 and may be electrically active.

The ablation probe 104 may further include a stick region 214, alternately referred to as a “tissue-loc” region, provided on the probe cannula 204 and a plug region 216 provided on the probe cannula 204 distal to the stick region, at or near the antenna 208. The stick and plug regions 214, 216 may be defined as portions of the probe cannula 204. The stick and plug regions 214, 216 will be discussed in more detail below.

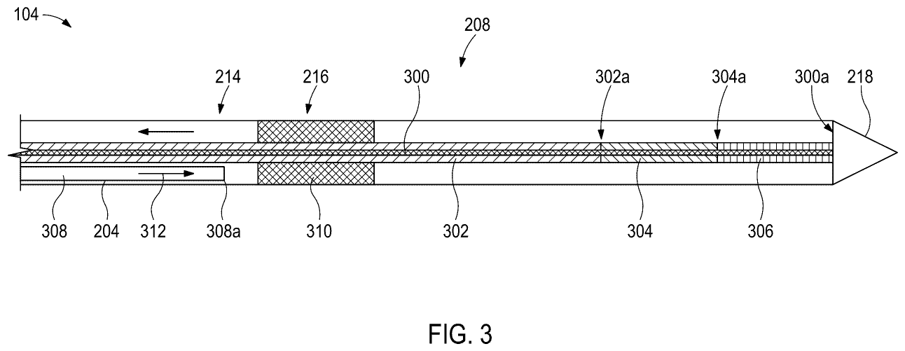

FIG. 3 is an enlarged, cross-sectional view of the probe cannula 204 and antenna 208 of the ablation probe 104, in accordance with at least one aspect of the present disclosure. The ablation probe 104 may include an inner conductor 300 and an outer conductor 302 extending through the probe cannula 204 and the antenna 208, with the outer conductor 302 being positioned about (around) the inner conductor 300. As illustrated, a distal end 300a of the inner conductor 300 extends distal to (beyond) a distal end 302a of the outer conductor 302. The inner and outer conductors 300, 302 may be made of a conductive material that allows current to be transmitted along their lengths thereof. The inner and outer conductors 300, 302 may be made of a metal, for example, such as stainless steel, silver, copper, brass or aluminum, or alloys thereof.

The ablation probe 104 may further include an insulator 304 extending from the distal end 302a of the outer conductor 302 and positioned about (around) the inner conductor 300. The insulator 304 may be made of a variety of non-conductive materials including, but not limited to, a ceramic or a polymer, such as polyamide, linear polyethylene (PE), polyethylene terephthalate (PET), polytetrafluoroethylene (PTFE) isotactic polypropylene (PP), or a polymer in the polyaryletherketone (PAEK) family, such as polyetheretherketone (PEEK), as examples.

The ablation probe 104 may further include a conductor load 306 extending from a distal end 304a of the insulator 304 and positioned about (around) the inner conductor 300. Accordingly, the insulator 306 axially interposes the outer conductor 302 and the conductor load 306. The conductor load 306 may be made of a metal, such as stainless steel, silver, copper, brass or aluminum, alloys thereof, or any combination thereof. The conductor load 306 may serve as a load point and may include a longitudinal length that is tuned to match the dielectric properties of the surrounding tissue.

The ablation probe 104 may further include a cooling tube 308 extending through the probe cannula 204 and terminating at the stick region 214. The cooling tube 308 may be fluidically coupled to the cooling tube 212 (FIG. 2) such that the cooling tube 308 may convey coolant 312 from the coolant source 107 (FIG. 1) and the cooling tube 212 (FIG. 2) to the stick region 214. A user may provide an input to the handle 202 (FIG. 2) and/or the GUI 120 (FIG. 1) to control flow of the coolant 312 through the cooling tube 308 to regulate a temperature of the antenna 208, the ablation zone 210, and/or the stick region 214, as will be discussed in more detail below. The cooling tube 308 may be made of a polymer, such as polyamide, linear polyethylene (PE), polyethylene terephthalate (PET), polytetrafluoroethylene (PTFE) isotactic polypropylene (PP), or a polymer in the polyaryletherketone (PAEK) family, such as polyetheretherketone (PEEK), as examples.

The stick region 214 is designed to attain and maintain a temperature that accommodates adherence of a tissue region onto the surface of the stick region 214. More specifically, the stick region 214 may operate as an anchoring element that freezes the interface between the stick region 214 and the adjacent tissue, thereby sticking (maintaining, locking, etc.) the antenna 208 in place. In operation, coolant 312 from the coolant source 107 (FIG. 1) may be conveyed to the stick region 214 by way of the cooling tube 212 (FIG. 2) and expelled from a distal end 308a of the cooling tube 308. Upon expulsion from the distal end 308a of the cooling tube 308, the pressure of the coolant 312 quickly decreases, thereby causing the coolant 312 to correspondingly decrease in temperature (the Joule-Thompson effect). The decreased temperature of the coolant 312 causes the temperature of the stick region 214 to decrease, and the coolant 312 is recirculated back along the probe cannula 204, such as to a coolant sink, for example.

Once a threshold “low” temperature is reached at the stick region 214, contact with adjacent tissue causes the tissue to adhere (stick or couple) to the stick region 214, thereby resulting in attachment of the energy delivery device 104 to the tissue. During ablation, as the tissue warms, the antenna 208 remains secured to the tissue region due to tissue desiccation and charring. The stick region 214 may be made of any material able to attain and maintain a temperature such that contact with tissue results in adherence of the tissue onto the stick region 214. Example materials for the stick region 214 include, but are not limited to, a metal.

With reference now to FIGS. 1 and 3, the controller 114 may control a state of one or more of the the valves 113, thereby controlling an amount of coolant 312 provided to the stick regions 214 of the ablation probes 104. Tissue may not adhere to the stick region 214 unless a threshold flow rate and/or pressure of coolant 312 is conveyed through the cooling tube 308 to the stick region 214. For instance, the valves 113 may be transitionable between a first state and a second state. The first state may be a first, partially opened state of the valve 113, where the valve 113 is opened a first amount, and the second state may be a second, partially opened state or the open state of the valve 113, where the valve 113 is opened a second amount greater than the first amount. The first and second states of the valves 113 may be stored in the memory 117.

In the first state, a first flow rate and/or pressure of coolant 312 may be conveyed through the cooling tube 308 to the stick region 214. The first flow rate and/or pressure of coolant 312 may be insufficient to cause tissue to adhere to the stick region 214, thereby allowing the stick region 214 to move relative to the tissue positioned thereagainst. In the second state, a second flow rate and/or pressure of coolant 312 greater than the first flow rate and/or pressure of coolant 312 may be conveyed through the cooling tube 308 to the stick region 214. The second flow rate and/or pressure of coolant 312 may be sufficient to cause tissue to adhere to the stick region 214, thereby preventing the stick region 214 from moving relative to the tissue positioned thereagainst.

The ablation probe 104 may further include a seal 310, which may define the plug region 216 on the probe cannula 204. As illustrated, the seal 310 may be provided distal to the distal end 308a of the cooling tube 308 and the stick region 214 and otherwise interposing the stick region 214 and the antenna 208. The seal 310 may be configured to prevent a reduction in temperature resulting from the cooled probe cannula 204 and the stick region 214 from affecting (e.g., reducing) the temperature within the antenna 208. Accordingly, the seal 310 separates interior portions of the ablation probe 104 to prevent cooling or heating of a portion or portions of the probe 104 while permitting cooling or heating of other portions. The seal 310 may be made of an insulative material capable of being in contact with a material or region having a low temperature without having its temperature significantly reduced. Example insulative materials for the seal 310 include, but are not limited to, a synthetic polymer (e.g., polystyrene, polyicynene, polyurethane, polyisocyanurate), aerogel, fiberglass, cork, or any combination thereof.

Additional information regarding the ablation probe 104, such as the construction and function thereof, is described in U.S. Pat. No. 11,638,607, entitled “ENERGY DELIVERY SYSTEMS AND USES THEREOF”, which issued on May 2, 2023, the contents of which are hereby incorporated by reference in their entirety herein.

With continued reference to FIGS. 1 and 3, the controller 114 may be operable to use the coolant source 107 and the coolant stored therein to reduce undesired heating within and along the ablation probes 104. In particular, the controller 114 may control conveyance (flow) of the coolant into and out of the cooling tube 308 via the valves 113. The controller 114 may also be configured to control conveyance of coolant to the stick region 214, as described herein above to thereby attain and maintain a temperature that accommodates adherence of tissue onto the surface of the stick region 214. In some embodiments, a user may provide an input to the controller 114, such as via the GUI 120. Based on the input, the controller 114 may control a state of one or more of the valves 113, as discussed herein above, thereby allowing the coolant source 107 to provide coolant to the stick region 214 via the cooling tubes 212, 308.

The controller 114 may also be operable to continuously or intermittently monitor the real-time temperature of the ablation probes 104. In such embodiments, the controller 114 may communicate with one or more temperature sensors (e.g., thermocouples) terminating at various points along the probe cannula 204 and/or the antenna 208 (FIG. 2) of the ablation probe 104. Consequently, localized temperature may be monitored at several points along the antenna 208 to estimate ablation status, cooling status, or safety checks. In some applications, monitoring the temperature at several points along the antenna 208 may help determine the geographical characteristics of the ablation zone 210, such as diameter, depth, length, density, width, etc., based upon the tissue type, and the amount of power used in the ablation probe 104. In other embodiments, or in addition thereto, the temperature may be measured not only at specific points along the probe cannula 204, but continuously along its entire length.

In some embodiments, the probe cannula 204 includes a plurality of temperature sensors. A first temperature sensor may be placed at, or slightly proximal to, the antenna 208 to provide real-temperature measurements of the tissue being heated by the antenna 208. A second temperature sensor may be placed at, or adjacent to, the stick region 214 to provide real-time temperature measurements of the tissue that is being cooled, and thus adhered to, the stick region 214. A third temperature sensor may be located proximal to the first and second temperature sensors along the cannula 204, such as at the point of entry into the skin, to provide real-time measurements of the patent's skin. The control system 102 can receive the temperature measurements from the first, second, and third sensors to control the coolant systems and cooling fluids from the controller 114 to the stick region 214 and/or other cooling systems of the energy delivery device 104.

The controller 114 may also be operable to monitor the temperature of a tissue region (e.g., tissue being treated, surrounding tissue). This may prove advantageous in helping to determine the status of the procedure (e.g., the end of the procedure). The controller 114 may communicate with the plurality of temperature sensors to provide real-time temperature information to a user and display such measurements on the GUI 120. In at least one embodiment, based on the temperature data obtained by the controller 114, the controller 114 may be configured to autonomously adjust operation of the system 100 appropriately.

As referenced above, proper positioning of an ablation device, like ablation probe 104, within a patient is critical to ensuring that energy emitted therefrom affects intended targets, such as tumors, while avoiding healthy tissue and critical structures, such as arteries, as much as possible. The positioning of the ablation devices may be determined using doppler ultrasound, which uses the relative speed of sound coming back to an ultrasound transducer (e.g., where the imaging device 119 (FIG. 1) is an ultrasound transducer) to determine motion, which can in turn be used to determine a position of the ablation probe 104. However, the echogenicity of the ablation probe 104 may be limited due to smooth surfaces thereof, like the smooth surfaces of the probe cannula 204, which may reflect the sound waves away from the ultrasound transducer 119 instead of scattering the sound waves back thereto. The systems and methods described herein for detecting a position of the ablation probe 104 using doppler ultrasound help remedy this shortcoming.

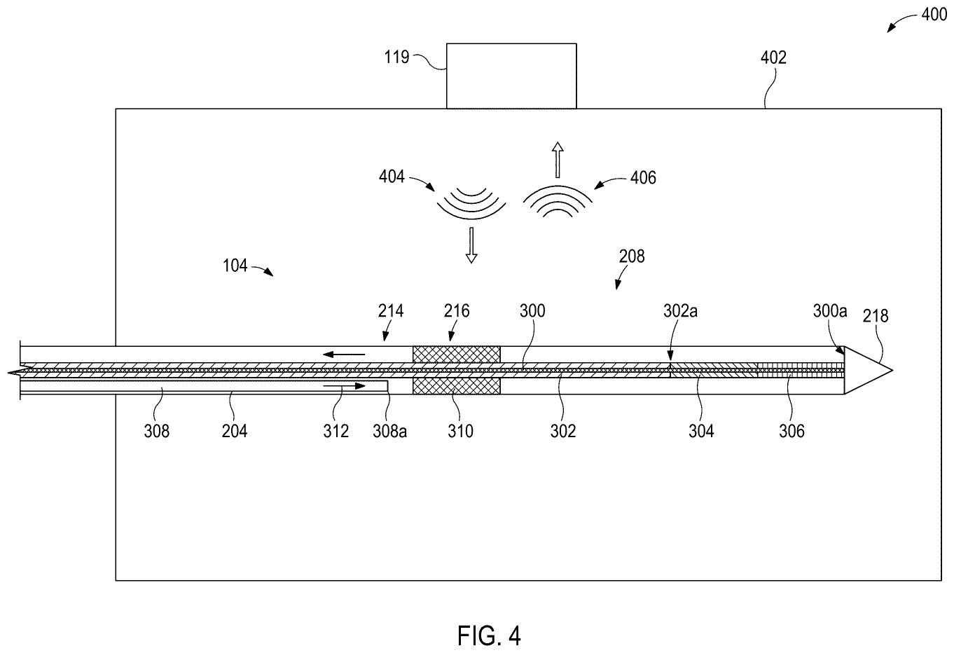

FIG. 4 is a schematic diagram of the ablation probe 104 of FIG. 3 percutaneously inserted into a patient 400, in accordance with at least one aspect of the present disclosure. As illustrated, the ultrasound transducer 119 may be placed (positioned) on the skin 402 of the patient 400 and may be operable to emit a sound wave or waves 404 into the patient 400, such as towards the ablation probe 104.

The inventors have identified that conveyance (flow) of coolant 312 through the cooling tube 308 to the stick region 214 may cause portions of the ablation probe 104, such as the probe cannula 204 and/or the antenna 208, to vibrate. When imaged with doppler ultrasound, these vibrations create what is known as a “twinkling artifact”, as will be discussed in more detail below.

With reference to FIGS. 1 and 4, when a user desires to know the position of the ablation probe 104 within the patient 400 prior to energizing the ablation probe 104, the user may provide an input to the controller 114, such as via the GUI 120. Based on receiving the input, the controller 114 may transition the valve 113 associated with the desired the ablation probe 104 to the first state, which, as described above, is a state in which the valve 113 is opened a first amount to allow conveyance of a first flow rate and/or pressure of coolant 312 through the cooling tube 308 to the stick region 214, but is insufficient to cause tissue to adhere to the stick region 214. Importantly, however, the first state may be sufficient to induce at least a portion of the ablation probe 104, such as the probe cannula 204 and/or the antenna 208, to vibrate.

Once the valve 113 is transitioned to the first state, the controller 114 may cause the ultrasound transducer 119 to emit sound waves 404 toward the ablation probe 104. Alternatively, the controller 114 may cause the ultrasound transducer 119 to emit the sound waves 404 based on a user providing an input to the GUI 120, or may emit the sound waves 404 periodically or continuously based on the ultrasound transducer 119 being powered on. The ultrasound transducer 119 may further receive reflected sound waves 406 based on the sound waves 404 reflecting off various anatomical structures, the ablation probe 104, etc. The controller 114 may receive the reflected sound waves 406 and display, on the GUI 120, an image according to (representative of) the reflected sound waves 406.

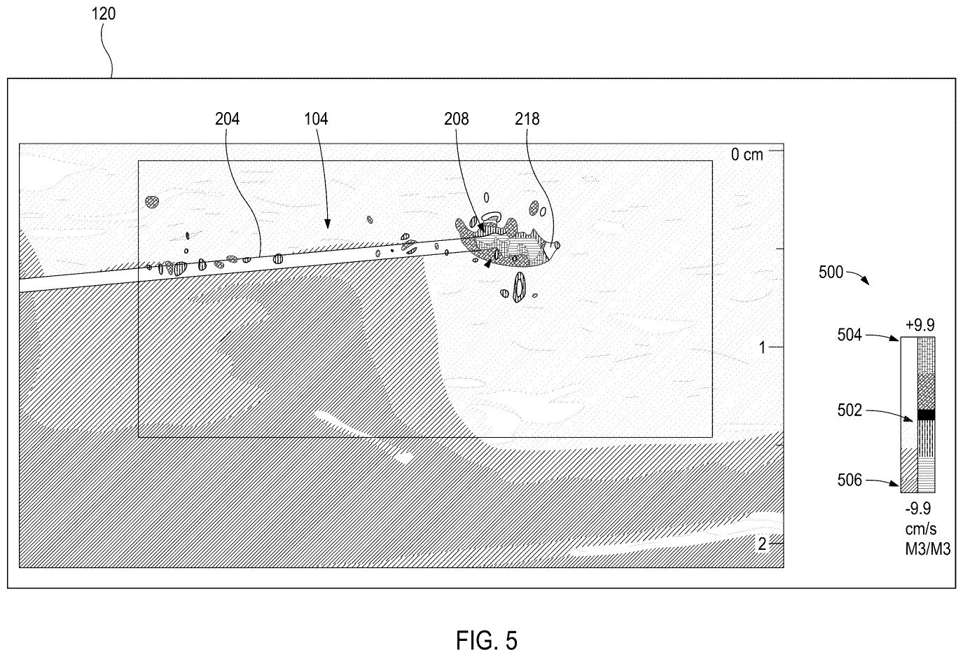

Referring now to FIG. 5, with continued reference to FIGS. 1 and 4, illustrated is an example image produced by the ultrasound transducer 119 based on the reflected sound waves 406 and displayed on the GUI 120 by the controller 114, according to the principles of the present disclosure. As referenced above, conveyance of coolant 312 through the cooling tube 308, such as with the valve 113 in the first state, may induce vibrations in portions of the ablation probe 104. When imaged with the ultrasound transducer 119, the reflected sound waves 406 may create what is known as a “twinkling artifact”.

More specifically, the controller 114 may display, on the display 120, a scale 500 with a center 502, a first end 504, and a second end 506 opposite the first end 504. The controller 114 may display the scale 500 in a plurality of colors with the center 502 being a first color, such as black, the first end 504 being a second color, such as yellow, and the second end 506 being a third color, such as cyan. The controller 114 may further display a first gradient colors between the center 502 and the first end 504 (e.g., red transitioning towards yellow) and a second gradient of colors between the center 502 and the second end 506 (e.g. dark blue transitioning toward cyan). A color on the scale 500 may correspond to a relative speed detected by the controller 114 via the ultrasound transducer 119.

Based on the reflected sound waves, the controller 114 may determine a speed of portions of the ablation probe 104 as a result of the induced vibrations. The speed may range from a first maximum speed toward the ultrasound transducer 119 and a second maximum speed away from the ultrasound transducer 119. The controller 114 may display portions of the ablation probe 104 moving at the first maximum speed (toward the ultrasound transducer 119) as the second color (yellow) shown on the first end 504 of the scale 500. Similarly, the controller 114 may display portions of the ablation probe 104 moving at the second maximum speed (away from the ultrasound transducer 119) on the display 120 as the third color (cyan) shown on the second end 506 of the scale 500. Furthermore, the controller 114 may display portions of the ablation probe 104 not moving as the first color (black) and portions of the ablation probe 104 moving at speeds toward and away from the ultrasound transducer 119 in colors according to their corresponding color on the scale 500. For example, a portion of the ablation probe 104 moving at a speed toward the ultrasound transducer 119 that is less than the first maximum speed, but greater than 0, may be displayed as orange from the first gradient of colors. Accordingly, a user visualizing the display 120 may see a plurality of colors from the scale 500 due to the vibrating ablation probe 104 (e.g., a “twinkling artifact”). This twinkling artifact increases the ability of the user to identify the position of the ablation probe 104 as compared to doppler ultrasound imaging of ablation probes that are not vibrating.

Furthermore, as referenced above, the controller 114 may place the valve 113 in the first state, which allows conveyance (flow) of coolant 312 through the cooling tube 308 that enables the user to better visualize the ablation probe 104, as described above, but does not cause tissue to adhere to the stick region 214 (e.g., the flow is slow enough that the temperature remains above a freezing point). Accordingly, with the valve 113 in the first state, a user may be able visualize the ablation probe 104 on the GUI 120, while also being able to reposition (move) the ablation probe 104, such as from a first or “undesired” position to a second or “desired”position.

Once the user has moved the ablation probe 104 to the desired position, the user may provide an input to the controller 114 to transition the valve 113 associated with the ablation probe 104 from the first state to the second state, in which the valve 113 is opened a second amount (e.g., to an increased degree) to convey (flow) the coolant 312 through the cooling tube 308 to the stick region 214 at a second flow rate and/or pressure that is sufficient to cause tissue to adhere to the stick region 214. Accordingly, once the user is satisfied with the position of the ablation probe 104, the user may increase the flow of the coolant, thereby causing tissue in contact with the stick region 214 to couple (“stick”) thereto and preventing the ablation probe 104 from moving relative to the tissue. The user may then provide an input to the controller 114 to energize the ablation probe 104, as described elsewhere herein.

Accordingly, the foregoing system increases visualization of the ablation probe 104 using doppler ultrasound by way of conveyance (flow) of coolant through the ablation probe 104. Furthermore, the controller 114 may control a flow rate and/or pressure of the coolant conveyed to the ablation probe 104 over time, with the controller 114 providing a first or “lesser” flow rate when a user desires to visualize and move the ablation probe 104, and a second or “greater” flow rate when a user desires to “lock”the ablation probe 104 in place.

In some embodiments, the controller 114 may be transitionable between a first or “pulsed” state and a second or “freezing” state. In the first state, the controller 114 may control the valve 113 to allow coolant 312 to be pulsed through the cooling tube 308. For instance, the controller 114 may transition the valve 113 between the closed state (e.g., 0% open) and the partially opened state (e.g., 10%, 25%, 50%, 75%, or 90%, for example) or the open state (e.g., 100%) at a predefined frequency, which may be stored in the memory 117. The pulsed flow of the coolant 312 through the cooling tube 308 may induce (cause) vibrations in the ablation probe 104 that are sufficient to be able to view the ablation probe 104 with doppler ultrasound, as discussed above, but insufficient to decrease the temperature of the ablation probe 104 to a point where tissue would stick (couple, adhere) to the stick region 214, thereby allowing the ablation probe 104 to be moved and repositioned.

In the freezing state, the controller 114 may control the valve 113 to allow coolant 312 to be conveyed through the cooling tube 308 at a flow rate and/or pressure that is sufficient to decrease the temperature of the ablation probe 104 to a point where tissue sticks (couples, adheres) to the stick region 214. Accordingly, once satisfied with the position of the ablation probe 104, a user may provide an input to the controller 114 to transition from the pulsed state to the freezing state.

As will be appreciated, operating the controller 114 in the pulsed state may reduce the amount of coolant 312 required while still inducing vibrations in the ablation probe 104 to increase visualization of the ablation probe 104 using doppler ultrasound.

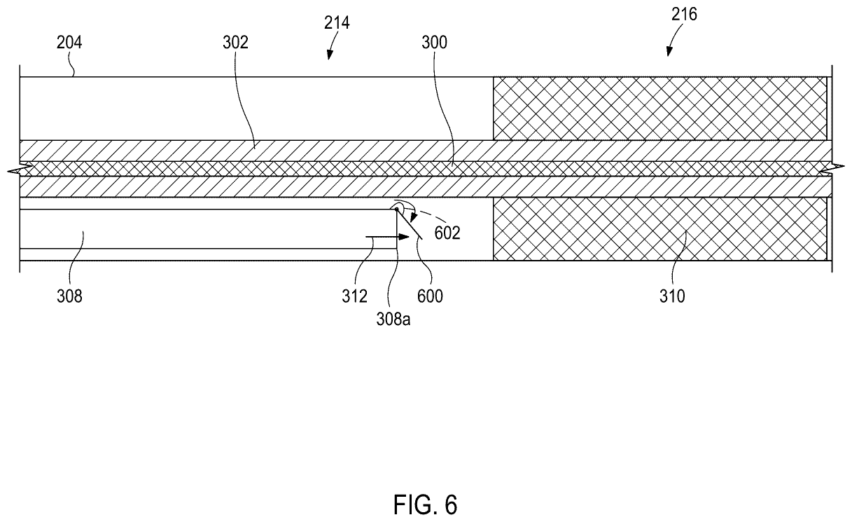

FIG. 6 is an enlarged view of the probe cannula 204 of the ablation probe 104 of FIG. 3, in accordance with one or more additional aspects of the present disclosure. As illustrated, the cooling tube 308 may include a check valve 600 pivotably coupled to a distal end 308a of the cooling tube 308. The check valve 600 may be transitionable between a first or “open” state, as shown in FIG. 6, in which the check valve 600 permits coolant 312 to exit the distal end 308a of the cooling tube 308, and a second or “closed” state, in which the check valve 600 prevents coolant 312 from exiting the distal end 308a of the cooling tube 308.

In some embodiments, the check valve 600 may comprise a flapper valve that includes a torsion spring 602 that naturally biases the check valve 600 toward the closed state. The spring bias of the torsion spring 602 may be overcome when a threshold (predetermined) flow rate of coolant and/or a threshold (predetermined) pressure of coolant 312 is circulated (flows) through the cooling tube 308. This transitions the check valve 600 from the closed state to the open state. Once the flow or pressure of the coolant 312 circulating in the cooling tube 308 drops below the threshold flow rate or pressure, the torsion spring 602 may naturally bias the check valve 600 to the closed state.

In example operation, with reference to FIGS. 1 and 6, the controller 114 may pulse coolant 312 through the cooling tube 308, causing the check valve 600 to transition between the open and closed states, thereby inducing mechanical vibrations in the ablation probe 104. The resulting mechanical vibrations may be seen with doppler ultrasound, and thereby more accurately locating the ablation probe 104. More specifically, and similar to above, the controller 114 may be transitionable between the pulsed and freezing states. In the pulsed state, the controller 114 controls the valve 113 to allow coolant 312 to be pulsed through the cooling tube 308 at flow rate and/or pressure sufficient to overcome the bias of the spring 602, but insufficient to cause tissue to couple (freeze, adhere) to the stick region 214. For instance, the controller 114 may transition the valve 113 between the closed state (e.g., 0% open) and the partially opened state (e.g., 10%, 25%, 50%, 75%, or 90%) or the open state (e.g., 100%) at a predefined frequency, which may be stored in the memory 117. While the controller 114 is in the pulsed state, the ultrasound transducer 119 may operate to accurately image the location of the ablation probe 104, thereby allowing the user to move and reposition the ablation probe 104 to a desired location, as needed.

Once satisfied with the position of the ablation probe 104, a user may provide an input to controller 114 to transition from the pulsed state to the freezing state. In the freezing state, the controller 114 may control the valve 113 to allow coolant 312 to be conveyed through the cooling tube 308 at a flow rate and/or pressure that overcomes the bias of the spring 602 and that is sufficient to cause tissue to couple (freeze, adhere) to the stick region 214.

Accordingly, inclusion of the valve 600 and the torsion spring 602 may increase the amount of vibration induced in the ablation probe 104 when conveying coolant therethrough, thereby increasing the ability of the ablation probe 104 to be imaged using doppler ultrasound.

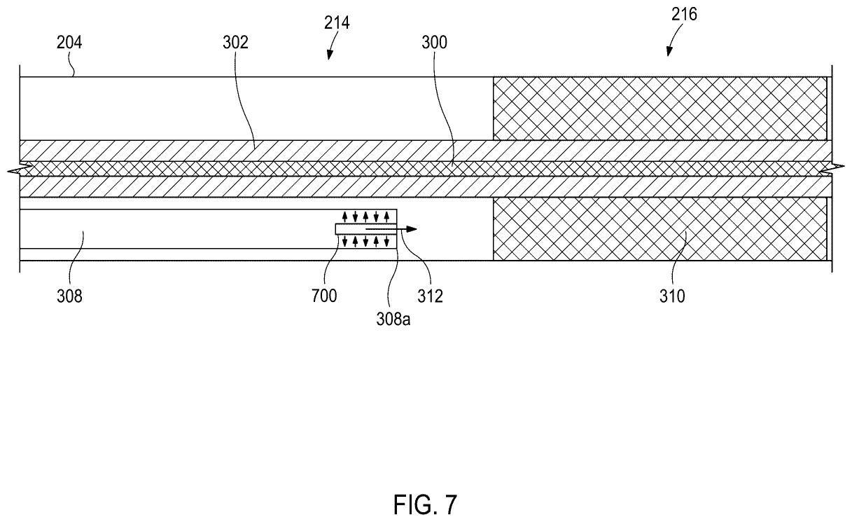

FIG. 7 is an enlarged view of the probe cannula 204 of the ablation probe 104 of FIG. 3, in accordance with one or more additional aspects of the present disclosure. In the illustrated embodiment, a strip of material 700 (e.g., a reed) may be arranged within the cooling tube 308 and otherwise positioned at or near the distal end 308a of the cooling tube 308. With the strip of material 700 present within the cooling tube 308, as the coolant 312 circulates through the cooling tube 308, the strip of material 700 may vibrate, thereby increasing the ability of the ablation probe 104 to be imaged using doppler ultrasound.

While one strip of material 700 is shown, the cooling tube 308 may include more than one strip of material 700. For instance, the cooling tube 308 may include a plurality of strips of material 700 serially or radially positioned along the flow path of the cooling tube 308, thereby increasing the amount of vibration induced in the ablation probe 104.

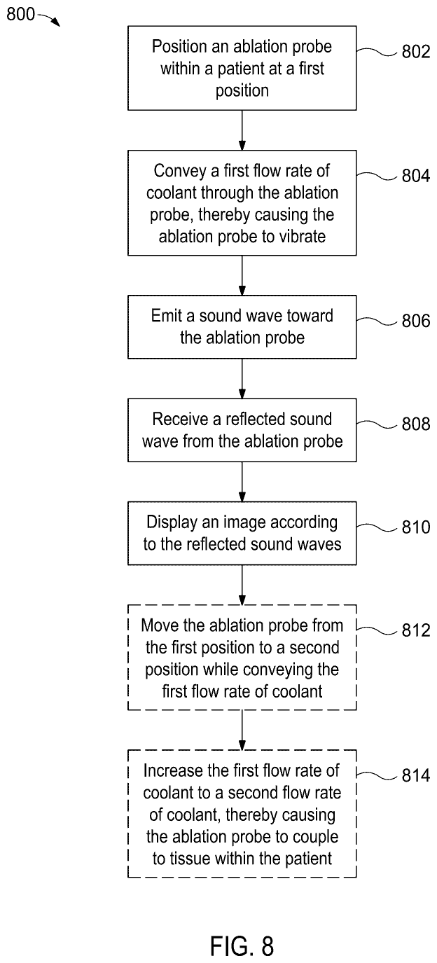

FIG. 8 is a schematic flow chart of an example method 800 of controlling the energy delivery system 100 of FIG. 1, according to at least one aspect of the present disclosure. The method 800 may be embodied as an algorithm stored in the memory 117 (FIG. 1) of the controller 114 (FIG. 1), and may be executable by the processor 115 (FIG. 1) of the controller 114.

With reference to FIGS. 1, 4, and 8, the method 800 may include positioning an ablation probe within a patient at a first position, as at step 802. For instance, a user may position the ablation probe 104 in a first position within the patient 400, like the position shown in FIG. 4.

The method 800 may further include conveying a first flow rate of coolant through the ablation probe, and thereby causing the ablation probe to vibrate, as at step 804. The user may desire to know the position of the ablation probe 104 within the patient prior to energizing the same. Accordingly, the user may provide an input to the controller 114, such as via the GUI 120, to transition the valve 113 associated with the ablation probe 104 to a state in which the valve 113 is opened to allow conveyance of coolant 312 through the cooling tube 308 to the stick region 214. The coolant may be conveyed through the cooling tube 308 at a flow rate and/or pressure insufficient to cause tissue to adhere (freeze, stick) to the stick region 214, but sufficient to induce at least a portion of the ablation probe 104 (e.g., the probe cannula 204, the antenna 208) to vibrate. The state of the valve 113 may be a partially opened state or a pulsed state.

The method 800 may further include emitting a sound wave toward the ablation probe, as at step 806. In some embodiments, the controller 114 may cause the ultrasound transducer 119 to emit sound waves 404 toward the ablation probe 104 once the valve 113 is transitioned, as at step 804. Alternatively, the controller 114 may cause the ultrasound transducer 119 to emit the sound waves 404 based on a user providing an input to the GUI 120, or may emit the sound waves 404 periodically or continuously based on the ultrasound transducer 119 being powered on.

The method 800 may further include receiving a reflected sound wave from the ablation probe, as at step 808. For instance, based on emitting sound waves toward the ablation probe at step 806, the ultrasound transducer 119 may receive reflected sound waves 406, which may then be received by the controller 114.

The method 800 may further include displaying an image corresponding to the reflected sound waves, as at step 810. For instance, the controller 114 may display, on the GUI 120, an image according to the reflected sound waves 406. This image may include an image that includes a “twinkling artifact”, as shown in FIG. 5, in which portions of the ablation probe 104 are shown as moving in a direction away from the ultrasound transducer 119, while other portions are shown as moving in a direction toward the ultrasound transducer 119. This twinkling artifact increases the ability of a user to identify the true position of the ablation probe 104 as compared to doppler ultrasound imaging of ablation probes that are not vibrating.

The method 800 may optionally include moving the ablation probe from a first position to a second position while conveying the first flow rate of coolant, as at step 812. For instance, based on viewing the twinkling artifact of the ablation probe 104 on the GUI 120, the user may desire to reposition (move) the ablation probe 104 from the first or “undesired” position to a second or “desirable” position. Accordingly, the user may move the ablation probe 104 within the patient 400 considering that the valve 113 is only allowing a flow rate and/or pressure of coolant 312 that does not “lock”the stick region 214 to tissue positioned thereagainst.

The method 800 may optionally further include increasing the first or “initial” flow rate of coolant to a second flow rate of coolant, thereby causing the ablation probe to couple to tissue within the patient, as at step 814. For instance, once the user has reached the desired position, the user may provide an input to the controller 114, such as via the GUI 120 to transition the valve 113 associated with the ablation probe 104 to a second or “freezing” state. In the freezing state, the valve 113 is opened to a degree that allows conveyance of coolant 312 through the cooling tube 308 to the stick region 214 at a flow rate and/or pressure sufficient to cause tissue to adhere to the stick region 214. Accordingly, once the user is satisfied with the position of the ablation probe 104, the user may increase the flow of the coolant, thereby causing tissue in contact with the stick region 214 to couple (“stick”) thereto and prevent the ablation probe 104 from moving relative to the tissue. The user may then energize the antenna 208 to ablate tissue, as described elsewhere herein.

Embodiments disclosed herein include:
A. A system comprising an ablation probe, a display, an ultrasound transducer, and a controller in operable communication with the display and the ultrasound transducer. The ablation probe comprises a shaft including a stick region, an antenna extending from the shaft, and a cooling tube extending within the shaft. The controller is operable to convey a first flow rate of coolant through the cooling tube sufficient to cause the ablation probe to vibrate, but insufficient to cause tissue to couple to the stick region, convey a second flow rate of coolant through the cooling tube sufficient to cause tissue to couple to the stick region, and while conveying the first flow rate of coolant through the cooling tube, emit, from the ultrasound transducer, a sound wave toward the ablation probe, receive, at the ultrasound transducer, a reflected sound wave from the ablation probe, and display, on the display, an image corresponding to the reflected sound wave.
B. A system comprising an ablation probe, a display, an ultrasound transducer, and a controller in operable communication with the display and the ultrasound transducer. The ablation probe comprises a shaft, an antenna extending from the shaft and operable to emit energy therefrom, and a cooling tube extending within the shaft. The controller is operable to provide a flow rate of coolant through the cooling tube while abstaining from emitting energy from the antenna, wherein the flow rate of coolant causes the ablation probe to vibrate, but fails to cause tissue to couple to the shaft, and while conveying the flow rate of coolant through the cooling tube, emit, from the ultrasound transducer, a sound wave toward the ablation probe, receive, at the ultrasound transducer, a reflected sound wave from the ablation probe, and display, on the display, an image corresponding to the reflected sound wave.
C. A method comprising positioning an ablation probe within a patient at a first position, conveying a first flow rate of coolant through the ablation probe, thereby causing the ablation probe to vibrate, emitting, from an ultrasound transducer, a sound wave toward the ablation probe, receiving, at the ultrasound transducer, a reflected sound wave from the ablation probe, displaying, on a display, an image corresponding to the reflected sound wave, and moving the ablation probe from the first position to a second position while conveying the first flow rate of coolant.

Each of embodiments A, B, and C may have one or more of the following additional elements in any combination: Element 1: wherein the controller is further operable to receive an input and increase the first flow rate of the coolant to the second flow rate based on the input. Element 2: further comprising a check valve coupled to a distal end of the cooling tube. Element 3: wherein the check valve is transitionable between an open state, in which the check valve permits coolant to exit the distal end of the cooling tube and a closed state, in which the check valve prevents coolant from exiting the distal end of the cooling tube, wherein movement of the check valve between the closed and open states causes the ablation probe to vibrate. Element 4: further comprising a torsion spring configured to bias the check valve toward the closed state. Element 5: further comprising a reed arranged at a distal end of the cooling tube. Element 6: wherein conveying the first flow rate of coolant through the cooling tube causes the reed to vibrate. Element 7: wherein the first flow rate comprises a pulsed flow rate. Element 8: wherein the flow rate of coolant is a first flow rate of coolant, and the controller is further operable to provide a second flow rate of coolant through the cooling tube, the second flow rate of coolant being sufficient to cause tissue to couple to the shaft. Element 9: wherein the controller is further operable to receive an input and increase the flow rate of coolant from the first flow rate to the second flow rate based on the input. Element 10: further comprising a check valve coupled to a distal end of the cooling tube. Element 11: further comprising a spring configured to bias the check valve to the closed state. Element 12: further comprising a reed arranged at a distal end of the cooling tube. Element 13: wherein conveying the first flow rate of coolant through the cooling tube causes the reed to vibrate. Element 14: further comprising increasing the first flow rate of the coolant to a second flow rate of coolant, and thereby causing the ablation probe to couple to tissue within the patient. Element 15: further comprising transitioning a check valve coupled to the ablation probe between open and closed states while conveying the first flow rate of coolant. Element 16: further comprising vibrating a reed coupled to the ablation probe while conveying the first flow rate of coolant.

By way of non-limiting example, exemplary combinations applicable to A, B, and C include: Element 1 with Element 2; Element 1 with Elements 2 and 3; Element 1 with Elements 2-4; Element 1 with Element 5; Element 1 with Elements 5 and 6; Element 1 with Element 7; Element 2 with Element 3; Element 2 with Elements 3 and 5; Element 2 with Elements 3 and 4; Element 2 with Element 5; Element 2 with Elements 5 and 6; Element 2 with Element 7; Element 5 with Element 6; Element 1 with two or more of Elements 2-7; Element 2 with two or more of Elements 1 and 3-7; Elements 2 and 3 with two or more of Elements 1 and 4-7; Elements 2-4 with two or more of Elements 1 and 5-7; Element 5 with two or more of Elements 1-4, 6, and 7; Elements 5 and 6 with two or more of Elements 1-4 and 7; Element 7 with two or more of Elements 1-6; Element 8 with Element 9; Element 8 with Element 10; Element 8 with Elements 10 and 11; Element 8 with Element 12; Element 8 with Elements 12 and 13; Elements 8 and 9 with Element 10; Elements 8 and 9 with Elements 10 and 11; Elements 8 and 9 with Element 12; Elements 8 and 9 with Elements 12 and 13; Element 10 with Element 11; Element 10 with Element 12; Element 10 with Elements 12 and 13; Elements 10 and 11 with Element 12 Elements 10 and 11 with Elements 12 and 13; Element 12 with Element 13; Element 14 with Element 15; Element 14 with Element 16; Element 15 with Element 16; Element 14 with Elements 15 and 16.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A system, comprising:
an ablation probe, comprising:
a shaft including a stick region;
an antenna extending from the shaft; and
a cooling tube extending within the shaft;
a display;
an ultrasound transducer; and
a controller in operable communication with the display and the ultrasound transducer, wherein the controller is operable to:
convey a first flow rate of coolant through the cooling tube sufficient to cause the ablation probe to vibrate, but insufficient to cause tissue to couple to the stick region;
convey a second flow rate of coolant through the cooling tube sufficient to cause tissue to couple to the stick region; and
while conveying the first flow rate of coolant through the cooling tube:
emit, from the ultrasound transducer, a sound wave toward the ablation probe;
receive, at the ultrasound transducer, a reflected sound wave from the ablation probe; and
display, on the display, an image corresponding to the reflected sound wave.

2. The system of claim 1, wherein the controller is further operable to:
receive an input; and
increase the first flow rate of the coolant to the second flow rate based on the input.

3. The system of claim 1, further comprising a check valve coupled to a distal end of the cooling tube.

4. The system of claim 3, wherein the check valve is transitionable between:
an open state, in which the check valve permits coolant to exit the distal end of the cooling tube; and
a closed state, in which the check valve prevents coolant from exiting the distal end of the cooling tube,
wherein movement of the check valve between the closed and open states causes the ablation probe to vibrate.

5. The system of claim 4, further comprising a torsion spring configured to bias the check valve toward the closed state.

6. The system of claim 1, further comprising a reed arranged at a distal end of the cooling tube.

7. The system of claim 6, wherein conveying the first flow rate of coolant through the cooling tube causes the reed to vibrate.

8. The system of claim 1, wherein the first flow rate comprises a pulsed flow rate.

9. A system, comprising:
an ablation probe, comprising:
a shaft;
an antenna extending from the shaft and operable to emit energy therefrom; and
a cooling tube extending within the shaft;
a display;
an ultrasound transducer; and
a controller in operable communication with the display and the ultrasound transducer, wherein the controller is operable to:
provide a flow rate of coolant through the cooling tube while abstaining from emitting energy from the antenna, wherein the flow rate of coolant causes the ablation probe to vibrate, but fails to cause tissue to couple to the shaft; and
while conveying the flow rate of coolant through the cooling tube:
emit, from the ultrasound transducer, a sound wave toward the ablation probe;
receive, at the ultrasound transducer, a reflected sound wave from the ablation probe; and
display, on the display, an image corresponding to the reflected sound wave.

10. The system of claim 9, wherein the flow rate of coolant is a first flow rate of coolant, and the controller is further operable to provide a second flow rate of coolant through the cooling tube, the second flow rate of coolant being sufficient to cause tissue to couple to the shaft.

11. The system of claim 10, wherein the controller is further operable to:
receive an input; and
increase the flow rate of coolant from the first flow rate to the second flow rate based on the input.

12. The system of claim 9, further comprising a check valve coupled to a distal end of the cooling tube.

13. The system of claim 12, further comprising a spring configured to bias the check valve to the closed state.

14. The system of claim 9, further comprising a reed arranged at a distal end of the cooling tube.

15. The system of claim 14, wherein conveying the first flow rate of coolant through the cooling tube causes the reed to vibrate.

16. A method, comprising:
positioning an ablation probe within a patient at a first position;
conveying a first flow rate of coolant through the ablation probe, thereby causing the ablation probe to vibrate;
emitting, from an ultrasound transducer, a sound wave toward the ablation probe;
receiving, at the ultrasound transducer, a reflected sound wave from the ablation probe;
displaying, on a display, an image corresponding to the reflected sound wave; and
moving the ablation probe from the first position to a second position while conveying the first flow rate of coolant.

17. The method of claim 16, further comprising increasing the first flow rate of the coolant to a second flow rate of coolant, and thereby causing the ablation probe to couple to tissue within the patient.

18. The method of claim 16, further comprising transitioning a check valve coupled to the ablation probe between open and closed states while conveying the first flow rate of coolant.

19. The method of claim 16, further comprising vibrating a reed coupled to the ablation probe while conveying the first flow rate of coolant.
